(19) United States
(12) Patent Application Publication
(10) Pub. No.: US 2002/0147217 A1
(43) Pub. Date: Oct. 10, 2002

(54) The treatment of sexual dysfunction with enantiomers

(75) Inventors: Neal R. Cutler, Los Angeles, CA (US); John Sramek, Irvine, CA (US)

Correspondence Address: MEDLEN & CARROLL, LLP, 101 HOWARD STREET, SUITE 350, SAN FRANCISCO, CA, 94105, US

(73) Assignee: R.T. Alamo Ventures I, L.L.C., Beverly Hills, CA (US)

(21) Appl. No.: 09/770,704

(22) Filed: Jan. 26, 2001

(51) Int. Cl. A61K 31/47

(52) U.S. Cl. 514/314

(57) ABSTRACT

Methods for treating specific patient groups for sexual dysfunction are provided. The methods of the present invention comprise the utilization of enantiomers of flosequinan, and pharmaceutical compositions comprising flosequinan enantiomers, in patients who are free of cardiac disease, who have not been treated with a drug that causes hypotensive effects, and/or who have not been given organic nitrites or nitrates. Flosequinan enantiomers, and pharmaceutical compositions comprising flosequinan enantiomers are administered to patients intranasally, orally, topically, and/or through respiratory inhalation.

### FIELD OF THE INVENTION

The present invention relates to methods for the treatment of sexual dysfunction in males and females (including but not limited to erectile dysfunction in males) in particular treatment groups. The methods of the present invention comprise the utilization of pharmaceutical compounds and compositions to patients who are free of symptoms of cardiac disease and who have not been treated with drugs which cause hypotensive effects, such as nitrites and nitrates.

### BACKGROUND

Impotence or erectile insufficiency is a widespread disorder that is thought to affect about twelve percent of adult men under age forty-five, about twenty percent of men at age sixty, and about fifty-five percent of men at age seventy-five.

There is more than one cause of erectile dysfunction. For example, erectile dysfunction can be psychological, resulting from anxiety or depression, with no apparent somatic or organic impairment. Such erectile dysfunction, which is referred to as “psychogenic,” is responsible for about fifteen to twenty percent of cases of impotence. In other cases, the erectile dysfunction is associated with atherosclerosis of the arteries supplying blood to the penis; such dysfunction is referred to as “arteriogenic” or “atherosclerotic.” About forty to sixty percent of cases of impotence are arteriogenic in origin.

In still other cases, there is leakage from veins in the penis such that sufficient pressure for an erection can be neither obtained nor maintained. This dysfunction is referred to as “venous leakage,” or “abnormal drainage.” This condition is often exacerbated by the presence of some arteriogenic dysfunction whereby the supply of blood to the penis is impaired. In still other cases, the dysfunction is associated with a neuropathy, such as nerve damage arising from, for example, surgery or a pelvic injury, in the nervous system affecting the penis. Such a dysfunction is referred to as “neurogenic” and this accounts for about ten to fifteen percent of cases of impotence.

There is also a high incidence of erectile insufficiency among diabetics, particularly those with insulin-dependent diabetes mellitus. Erectile dysfunction in diabetics is often classified as “diabetogenic,” although the underlying dysfunction is usually neurogenic associated with neuropathy, but may be arteriogenic or neurogenic and arteriogenic. About half of diabetic males suffer from erectile insufficiency, and about half of the cases of neurogenic impotence are in diabetics.

Additionally, erectile insufficiency is sometimes a side effect of certain drugs, such as beta-blockers that are administered to reduce blood pressure in persons suffering from hypertension, or drugs administered to treat depression or anxiety. Excessive alcohol consumption has also been linked to erectile insufficiency. These forms of erectile insufficiency may be regarded as a subset of neurogenic or psychogenic insufficiency.

A number of methods to treat impotence are available. These treatments include pharmacological treatments, surgery and, in cases of psychogenic dysfunction, psychological counseling is sometimes effective. Psychogenic impotence often can be cured by counseling coupled with a demonstration to the patient that he is capable of having a full erection by inducing such an erection once or a few times in the patients. Insufficiency due to excessive alcohol consumption is sometimes cured by reducing or elimination such consumption.

In rare cases, where the insufficiency is physical because of venous leakage, surgery can usually be employed to repair the venous lesion and thereby either cure the insufficiency or, if there remains an erectile insufficiency after repair of the venous lesion, render the insufficiency amenable to treatment by pharmacological methods. Also, penile implants, which provide a mechanical means to produce an erection sufficient for vaginal penetration, are widely used to treat impotence. In recent years, implants have been employed, especially in cases where pharmacological intervention is ineffective, which are usually cases of severe atherogenic impotence. Treatment of impotence with penile implants, however, entails serious disadvantages. Such treatment requires surgery and necessitates total destruction of the erectile tissues of the penis, forever precluding normal erection.

Pharmacological methods of treatment are also available. Such methods, however, have not proven to be highly satisfactory and can be accompanied by severe side-effects. Papaverine is now widely used to treat impotence, although papaverine is ineffective in overcoming impotence due, at least in part, to severe atherosclerosis. Papaverine is effective in cases where the dysfunction is psychogenic or neurogenic and severe atherosclerosis is not involved. Injection of papaverine, a smooth muscle relaxant, or phenoxybenzamine, a non-specific blocker and hypotensive, into a corpus cavemosum has been found to cause an erection sufficient for vaginal penetration. Also, in cases where severe atherosclerosis is not a cause of the dysfunction, intracavernosal injection of phentolamine, an α-adrenergic blocker, causes an erection sufficient for vaginal penetration. The resulting erection is one of significantly shorter duration than that induced by intracavernosal injection of papaverine or phenoxybenzamine and is of such short duration that satisfactory sexual relations are difficult or impossible.

Treatment of impotence with papaverine or phenoxybenzamine often results in priapism, a locking-up of an erection for a long period of time, typically a few hours and sometimes longer than twenty-four hours. Priapism is a serious, deleterious side effect of treatment of erectile insufficiency with these drugs. Beyond the embarrassment that may be caused for some men, priapism is usually painful, irreversibly damages erectile tissue, and, to be relieved, requires bleeding or pharmacological intervention, such as injection of a sympathomimetic drug, such as adrenaline.

Even if priapism does not occur with use of papaverine, such use is associated with a painful, burning sensation in the first two or so minutes after the injection and there are indications that repeated use of papaverine causes undesirable, extensive intracavernous fibrosis. Further, as indicated above, impotence arising from severe atherosclerosis is not susceptible to treatment with papaverine, phenoxybenzamine, phentolamine or papaverine together with phentolamine. In any case, phenoxybenzamine is not suitable for use in treating impotence because it is a carcinogen.

Sildenafil citrate (Viagra) has also been utilized as a pharmacological treatment for impotence. However, sildenafil citrate has a lack of specificity for its target, enzyme phosphodiesterase 5 (PDE5), and exerts a definite inhibition on the enzyme phosphodiesterase 6 (PDE6), located in the retina. It has been shown that the inhibition of PDE6 results in color vision defects as a side effect of treatment with sildenafil citrate. Furthermore, side effects such as flushing, headache, nasal congestion, and dyspepsia (heartburn) have also been associated with sildenafil citrate treatment of impotence. (See, Moreira et al., “Side-effect profile of sildenafil citrate (Viagra) in clinical practice,” Urology, 56(3): 474-76 (2000)).

Thus, although impotence is a ubiquitous problem, there are few satisfactory methods available for treating this disorder. Because of the relatively invasive intervention involved and the high failure rate of penile prostheses, surgical approaches provide unattractive alternatives. A safe pharmacological approach to the treatment of impotence is still to be achieved.

What is needed is a pharmaceutical that is effective but lacking in significant side effects.

### SUMMARY OF THE INVENTION

The present invention relates to methods for the treatment of sexual dysfunction in males and females (including but not limited to erectile dysfunction in males) in particular treatment groups. The methods of the present invention comprise the utilization of pharmaceutical compounds and compositions to patients who are free of symptoms of cardiac disease and who have not been treated with drugs which cause hypotensive effects, such as nitrites and nitrates. The compositions comprise purified enantiomers of flosequinan, including derivatives thereof. In one embodiment, said purified enantiomer of flosequinan is a (+) enantiomer. In another embodiment, said composition is substantially free of the (−) enantiomer of flosequinan.

In one embodiment, the method comprises a) providing: i) a subject (whether male or female) suffering from symptoms of sexual dysfunction; and ii) a pharmaceutical composition comprising a purified enantiomer of flosequinan, or a pharmaceutically acceptable salt thereof; and b) administering said pharmaceutical composition to the subject such that symptoms are reduced. A variety of such symptoms are contemplated, including but not limited to, poor blood flow to the sexual organs and/or failure to achieve orgasm. In one embodiment, the present invention contemplates administering said flosequinan to said male or female under conditions such that blood flow to the sexual organs of said male or female is improved.

It is not intended that the methods of the present invention be limited to a patient who is free from cardiac disease. However, in one embodiment, the patient is free from cardiac disease. In another embodiment, said patient is not free from cardiac disease. In one embodiment, the patient is male. In another embodiment, the patient is female. In one embodiment, said administering step is selected from the group consisting of intranasal and respiratory inhalation.

It is not intended that the methods of the present invention be limited to a patient who has been, or is being, treated with a drug that causes hypotensive effects. However, in one embodiment, the method comprises a) providing: i) a patient (whether male or female) suffering from symptoms of sexual dysfunction who is not being treated with a drug that causes hypotensive effects, and ii) a pharmaceutical composition comprising a purified enantiomer of flosequinan, or a pharmaceutically acceptable salt thereof; and b) administering flosequinan to the patient such that such symptoms are reduced. A variety of such symptoms are contemplated, including but not limited to, poor blood flow to the sexual organs and/or failure to achieve orgasm. In one embodiment, the present invention contemplates administering said flosequinan to said male or female under conditions such that blood flow to the sexual organs of said male or female is improved. In one embodiment, the patient is free from cardiac disease. In one embodiment, the patient is male. In another embodiment, the patient is female. In one embodiment, said administering step is selected from the group consisting of intranasal and respiratory inhalation. In one embodiment, said patient is being, or has been, treated in the past with a drug that causes hypotensive effects.

It is not intended that the methods of the present invention be limited to a patient who is being, or has been, treated with a nitrate or nitrite. However, in another embodiment, the method comprises a) providing: i) a patient (whether male or female) suffering from symptoms of sexual dysfunction who is not being treated with a nitrite or nitrate, and ii) a pharmaceutical composition comprising a purified enantiomer of flosequinan, or a pharmaceutically acceptable salt thereof; and b) introducing flosequinan to the patient such that such symptoms are reduced. A variety of such symptoms are contemplated, including but not limited to, poor blood flow to the sexual organs and/or failure to achieve orgasm. In one embodiment, the present invention contemplates administering said flosequinan to said male or female under conditions such that blood flow to the sexual organs of said male or female is improved. In one embodiment, the patient is free from cardiac disease. In one embodiment, the patient is male. In another embodiment, the patient is female. In one embodiment, said administering step is selected from the group consisting of intranasal and respiratory inhalation. In one embodiment, said patient has been, or is being, treated with a nitrite or nitrate. In one embodiment, said nitrate is selected from the group consisting of glyceryl trinitrate, isosorbide dinitrate, isosorbide-5′-mononitrate, and erythrityl tetranitrate.

In one embodiment, the method comprises providing: i) a male or female subject with erectile dysfunction, and ii) a purified enantiomer of flosequinan, or a pharmaceutically acceptable salt thereof; and introducing flosequinan to the male or female subject such that an erection (i.e. penile or clitoral) is produced.

In another embodiment, the method comprises providing: i) a male or female subject with erectile dysfunction, and ii) a pharmaceutical composition comprising a purified enantiomer of flosequinan, or a pharmaceutically acceptable salt thereof; and introducing the pharmaceutical composition to the male or female subject such that an erection (i.e. penile or clitoral) is produced.

It is not intended that the present invention be limited by the method of introduction of a purified enantiomer of flosequinan, or a pharmaceutically acceptable salt thereof. In one embodiment, the enantiomer of flosequinan is introduced into the male or female orally. It is believed that an oral dosage up to approximately 200 milligrams of a racemic mixture of flosequinan is an effective oral dosage. It is also believed that the oral administration of a purified of enantiomer of flosequinan is effective at even lower dosages (e.g. less than 200 mg). In one embodiment, the male or female is an adult human and the oral dosage of a purified enantiomer of flosequinan is in a single dose per day of up to approximately two hundred milligrams, more preferably, between approximately fifty to approximately seventy-five milligrams. In an even more preferred embodiment, a purified enantiomer of flosequinan is administered in a single oral dose per day of between approximately twenty and approximately fifty, and even more preferably, between approximately ten and approximately twenty milligrams per day. The administration of multiple dosages of a purified enantiomer of flosequinan are also contemplated.

In other embodiments, enantiomers of flosequinan are introduced cutaneously, transurethrally, by standard injection, intracavernosally, intranasally or through respiratory inhalation. In other embodiments, pharmaceutical compositions comprising a purified enantiomer of flosequinan, or a pharmaceutically acceptable salt thereof, is introduced cutaneously, transurethrally, by standard injection, intracavernosally, intranasally or through respiratory inhalation.

The present invention is not limited by the degree of response by the male subject. In one embodiment, the erection induced is sufficient for vaginal penetration.

Likewise, the present invention also contemplates the use of sexual stimulation in addition to the application of a pharmaceutical composition. For example, one embodiment comprises a) providing: i) a male, having a penis, with erectile dysfunction, ii) a purified enantiomer of flosequinan, or a pharmaceutically acceptable salt thereof, and iii) sexual stimulation; and b) introducing said flosequinan and sexual stimulation to the male such that an erection is produced.

In another embodiment, the method comprises a) providing: i) a male, having a penis, with erectile dysfunction, ii) a pharmaceutical composition comprising a purified enantiomer of flosequinan, or a pharmaceutically acceptable salt thereof, and iii) sexual stimulation; and b) introducing said pharmaceutical composition and sexual stimulation to the male such that an erection is produced.

In another embodiment, the present invention also contemplates the use of sexual stimulation in addition to the application of a pharmaceutical composition. For example, one embodiment comprises a) providing: i) a female, having a clitoris, with erectile dysfunction, ii) a purified enantiomer of flosequinan, or a pharmaceutically acceptable salt thereof, and iii) sexual stimulation; and b) introducing said flosequinan and sexual stimulation to the female such that an erection is produced.

In another embodiment, the method comprises a) providing: i) a female, having a clitoris, with erectile dysfunction, ii) a pharmaceutical composition comprising a purified enantiomer of flosequinan, or a pharmaceutically acceptable salt thereof, and iii) sexual stimulation; and b) introducing said pharmaceutical composition and sexual stimulation to the female such that an erection is produced.

Likewise, the present invention is not limited by the nature of the sexual stimulation. In one embodiment, the sexual stimulation is sexually explicit media. In another embodiment, the sexual stimulation involves manipulation of the penis, such as with vibration. In another embodiment, the sexual stimulation involves manipulation of the clitoris, such as with vibration or digital stimulation.

It is not intended that the present invention be limited by the nature of the formulation. In one embodiment, the present invention contemplates a formulation comprising a purified enantiomer of flosequinan, or derivative thereof, in a mixture comprising lactose.

### DESCRIPTION OF THE DRAWINGS

FIG. 1 depicts the respective HPLC column retention times and optical rotations of the enantiomers of flosequinan separated by the method provided in Example 2.

### DEFINITIONS

As used herein, the term “enantiomer” refers to stereoisomers of molecules that are non-superimposable mirror images of each other. Enantiomers have identical physical properties, such as melting points and boiling points, and also have identical spectroscopic properties. Enantiomers differ from each other with respect to their interaction with plane-polarized light and with respect to biological activity.

As used herein, the term “stereoisomer” refers to compounds that have their atoms connected in the same order but differ in the arrangement of their atoms in space. (e.g. cis-2-butane and trans-2-butane).

As used herein, the phrase “methylsulphinyl derivatives of quinolinone” refers to chemical compositions comprising quinolinone with a methylsulphinyl group attached. Examples include flosequinan (7-fluoro-1-methyl-3-(methylsulphinyl)-4(1H)-quinolone; 7-fluoro-1-methyl-3-(methylsufinyl)-4(1H)-quinolinone):

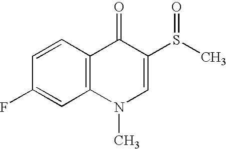

and sulfone metabolites of flosequinan:

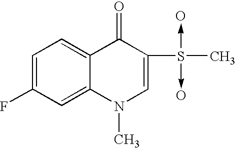

As used herein, the terms “purified enantiomer” and “purified enantiomer preparation” are meant to indicate a preparation (e.g. derived from a racemic mixture) wherein one enantiomer has been enriched over the other, and more preferably, wherein the other enantiomer represents less than 10%, and more preferably less than 5%, and still more preferably, less than 2% of the preparation.

As used herein, the term “racemic mixture” refers to a mixture of the two enantiomers of one compound. An ideal racemic mixture is one wherein there is a 50:50 mixture of both enantiomers of a compound such that the optical rotation of the (+) enantiomer cancels out the optical rotation of the (−) enantiomer.

As used herein, the phrase “pharmaceutically acceptable salts” or “a pharmaceutically acceptable salt thereof” refer to salts prepared from pharmaceutically acceptable non-toxic acids or bases including inorganic acids and bases and organic acids and bases. Since the compound of the present invention is basic, salts may be prepared from pharmaceutically acceptable non-toxic acids. Suitable pharmaceutically acceptable acid addition salts for the compound of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. Preferred acid addition salts are the chloride and sulfate salts.

As used herein, a patient who is “free from cardiac disease” and a patient who is “free from symptoms of cardiac disease” indicate that the patient has not been diagnosed with angina, myocardial infarction, congestive heart failure and that symptoms of angina, ischemia, myocardial infarction, and/or congestive heart failure have not been detected.

As used herein, “drugs that have hypotensive effects” are those drugs which, when administered, cause the patient's end-diastolic blood pressure to be reduced. Nitrates are commonly used drugs which have hypotensive effects.

As used herein, “nitrates” are compounds that contain the —NO₃— moiety. Nitrates typically used in the clinic are shown in Table 1.

As used herein, “nitrites” are compounds that contain the —NO₂— moiety. Nitrites typically used in the clinic are shown in Table 1.

As used herein, the term “erectile dysfunction” refers to certain disorders of the cavernous tissue of the penis and the associated facia which produce impotence, the inability to attain a sexually functional erection.

As used herein, “symptoms of erectile dysfunction” refers to any two of the following symptoms: penile flaccidity, lack of penile tumescence, lack of penile rigidity, and inability to produce an erection sufficient for vaginal penetration. Symptoms are “reduced” when the magnitude (e.g. intensity) or frequency of symptoms is reduced. It is not intended that the present invention be limited only to cases where the symptoms are eliminated. The present invention specifically contemplates treatment such that symptoms are reduced (and the condition of the patient is thereby “improved”), albeit not completely eliminated.

As used herein, “symptoms of sexual dysfunction” includes, but is not limited to, poor blood flow to the sexual organs and/or failure to achieve orgasm. Symptoms are “reduced” when the magnitude (e.g. intensity) or frequency of symptoms is reduced. It is not intended that the present invention be limited only to cases where the symptoms are eliminated. The present invention specifically contemplates treatment such that symptoms are reduced (and the condition of the patient is thereby “improved”), albeit not completely eliminated.

As used herein “standard injection” refers to the placement of a pharmaceutical composition into a subject (e.g., with a hypodermic needle). For example, such injection can be made subcutaneously, intravenously, intramuscularly, intracavernosally, etc.

As used herein, “intracavernosal” injection is injection into the corpus cavernosum of the penis.

As used herein, an “rection” refers to the condition of a penis whereby it is at least semi-rigid as opposed to being in a flaccid state.

As used herein, “by oral administration” refers to the introduction of a pharmaceutical composition into a subject by way of the oral cavity (e.g., in aqueous liquid or solid form).

As used herein, “cutaneously” refers to the introduction of a pharmaceutical composition into a subject by application to the surface of the skin such that the composition is absorbed into the subject. As used herein, “transurethrally” refers to the introduction of a pharmaceutical composition to the urethra of a subject such that the composition is absorbed into the subject.

As used herein, “intranasally” refers to the introduction of a pharmaceutical composition within the nasal cavity.

As used herein, “respiratory inhalation” refers to the introduction of a pharmaceutical composition within the respiratory tract.

As used herein, “sufficient for vaginal penetration” refers to the state of an erection such that the penis is capable of entering a vagina without manual manipulation.

As used herein, “sexual stimulation” refers to activity that would induce an erection in a male without erectile dysfunction (e.g., sexually explicit media, manual manipulation, vibration, live erotic entertainment, etc.)

As used herein, “sexually explicit media” refers to films, videos, books, magazines, etc. that depict sexual activity.

As used herein “single dosage” refers to a pharmaceutical composition of a formulation that is capable of achieving its intended effect in a single administration or application.

As used herein, the term “subject” refers to both humans and animals.

TABLE 1

| NONPROPRIETARY | | |
|---|---|---|
| NAMES AND TRADE | CHEMICAL | PREPARATIONS, USUAL DOSES, AND |
| NAMES | STRUCTURE | ROUTES OF ADMINISTRATION* |
| Amyl nitrite (isoamyl nitrite) | 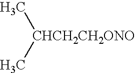 | Inh: 0.18 or 0.3 ml, inhalation |
| Nitroglycerin (glyceryl trinitrate; NITRO-BID, NITROSTAT, NITROL, NITRO-DUR, others) | 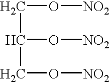 | T: 0.15 to 0.6 mg as needed S: 0.4 mg per spray as needed C: 2.5 to 9 mg two to four times daily |
| # B: 1 mg every 3 to 5 h O: 1.25 to 5 cm (½ to 2 in.), topically to skin every 4 to 8 h D: 1 disc (2.5 to 15 mg) every 24 h IV: 5 μg/min; increments of 5 μg/min | | |
| Isosorbide dinitrate (ISORDIL, SORBITRATE, DILATRATE, others) | 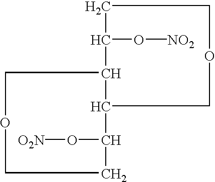 | T: 2.5 to 10 mg every 2 to 3 h T(C): 5 to 10 mg every 2 to 3 h T(O): 10 to 40 mg every 6 h C: 40 to 80 mg every 8 to 12 h |
| Isorbide-5-mononitrate (IMDUR, ISMO, others) | 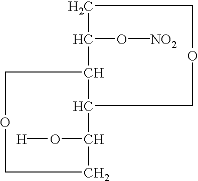 | T: 10 to 40 mg twice daily C: 60 mg daily |
| Erythrityl tetranitrate (CARDILATE) | 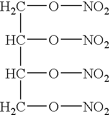 | T: 5 to 10 mg as needed T(O): 10 mg three times daily |

B buccal (transmucosal) tablet; C, sustained-release capsule or tablet; D, transdermal disc; Ink, inhalant; IV, intravenous injection; O, ointment; S, lingual spray; T, tablet for sublingual use; T(C), chewable tablet; T(O), oral tablet or capsule.

### DETAILED DESCRIPTION OF THE INVENTION

The present invention relates to methods for the treatment of sexual dysfunction in males and females (including but not limited to erectile dysfunction in males) in particular treatment groups. The methods of the present invention comprise the utilization of pharmaceutical compounds and compositions to patients who are free of symptoms of cardiac disease and who have not been treated with drugs which cause hypotensive effects, such as nitrites and nitrates. The compositions comprise enantiomers of flosequinan, including derivatives thereof.

In one embodiment a purified enantiomer of flosequinan is administered. Importantly, flosequinan enantiomers may potentiate the hypotensive effects of nitrates, and its administration to patients who are concurrently using organic nitrates in any form may be contraindicated. It is contemplated that an enantiomer of flosequinan be administered cutaneously, transurethrally, by standard injection, intracavemosally, intranasally or through respiratory inhalation, although it is not intended that the methods of the present invention be limited to the mode of administration of an enantiomer of flosequinan.

In other embodiments, a pharmaceutical composition comprising a purified enantiomer of flosequinan is administered. It is contemplated that said pharmaceutical composition be administered cutaneously, transurethrally, by standard injection, intracavemosally, intranasally or through respiratory inhalation, although it is not intended that the methods of the present invention be limited to the mode of administration of said pharmaceutical composition.

In one embodiment, the present invention contemplates the use of compositions that are effective to induce an erection in a human male suffering from impotence of any origin, other than anatomical deficiencies (i.e., lacking a penis or a significant portion thereof) that preclude an erection sufficient for vaginal penetration. In particular, these compositions may be used to induce an erection in a male suffering from impotence caused by severe atherosclerosis, and also impotence that is neurogenic or psychogenic in origin. The compositions utilized in the methods of the present invention comprise purified enantiomers of flosequinan, including derivatives thereof.

Although the present invention is not limited by a specific means of producing enantiomers of flosequinan, methods of producing a racemic mixture of flosequinan, are set forth in U.S. Pat. Nos. 5,079,264 and 5,011,931 to MacLean et al., hereby incorporated by reference. Moreover, a means of resolving the enantiomers of flosequinan is set forth in Morita et al., “Synthesis and Absolute Configuration of the Enantiomers of 7-Fluoro-1-methyl-3-(methylsulfinyl)-4(1H)-quinolinone (Flosequinan),” Chem. Pharm. Bull., 42(10): 2157-2160 (1994), hereby incorporated by reference.

Resolution of the (+) and (−) Enantiomers of Flosequinan

The present invention contemplates the resolution of the (+) and (−) enantiomers of flosequinan. Many organic compounds, including flosequinan, exist in optically active forms (i.e., they have the ability to rotate the plane of plane-polarized light). In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory (rotates to the left). A compound prefixed with (+) or d is dextrorotatory (rotates to the right). For a given chemical structure, these compounds, called “stereoisomers,” are identical except that they are mirror images of one another. A specific stereoisomer may also be referred to as an “enantiomer.

Stereochemical purity is of importance in the field of pharmaceuticals, where 12 of the 20 most prescribed drugs exhibit chirality. A case in point is provided by the L-form of the beta-adrenergic blocking agent, propranolol, which is known to be 100 times more potent than the D-enantiomer. Furthermore, optical purity is important since certain isomers may actually be deleterious rather than simply inert. For example, it has been suggested that the D-enantiomer of thalidomide was a safe and effective sedative when prescribed for the control of morning sickness during pregnancy, while the corresponding L-enantiomer has been believed to be a potent teratogen.

The present invention is not limited by any specific means of resolving the (+) and (−) enantiomers of flosequinan to obtain a purified enantiomer of flosequinan. In one embodiment, said enantiomers are resolved as follows. A racemic mixture of flosequinan is subjected to high-performance liquid chromatography (HPLC) over a Chiracel OD column (Chiral Technologies, Exton, Pa.) at a flow rate of 1.0 ml/minute. A mobile phase comprising methanol is utilized, resulting in the resolution of a distinct peak for each enantiomer. The resolved (+) and (−) enantiomers of flosequinan are eluted with methanol with an optical purity greater than 99%.

While it is not necessary to understand any particular mechanism to carry out the present invention, it is believed that in some circumstances, enantiomers of flosequinan can act as a direct-acting vasodilator to relax the corpus cavernosum smooth muscle cells, which in turn increases blood flow into the cavernosa space. This then leads to increased cavernosa pressure to produce an erect penis.

The action of the enantiomers of flosequinan in the body are not precisely understood. Its activity in the body is attributed to flosequinan itself, as well as its sulfone metabolite. It has been reported to be useful to some degree in the treatment of heart failure. (See Kelso et al., J. Cardiovasc. Pharmacol. 25: 376 (1995)). However, its action appears to have little effect in patients with end-stage failure (Perreault et al., Br. J. Pharmacol. 106: 511 (1992)) and does not affect mortality or arrhythmias following coronary artery ligation (See, Jones et al., Br. J. PharmacoL 108: 1111 (1993)).

Likewise, flosequinan has been reported to be a selective inhibitor of phosphodiesterase III (PDE3). (See, Gristwood et al., Br. J. Pharmacol., 105: 985 (1992); Frodsham et al., Eur. J. Pharmacol. 211: 383 (1992)). It has been found that inhibitors of PDE3 cause vasodilation leading to a concomitant reduction in arterial pressure. (See, Shiraishi et al., “Effect of cilostazol, a phosphodiesterase type III inhibitor, on histamine-induced increase in [Ca²⁺]ᵢ and force in middle cerebral artery of the rabbit,” Br. J. Pharmacol., 123: 869-878 (1998)). Moreover, it has also been shown that PDE3 is present in human corpus cavernosum tissue. (See, Ellis & Terrett, PCT Application No. WO 94/28902). However, reports concerning phosphodiesterase inhibition of flosequinan, as relevant to its efficacy in heart failure, is questionable. Thus, the application of flosequinan to particular purposes in the body is not well-characterized and must be determined empirically.

It is not intended that the present invention be limited to any particular mechanism to reduce symptoms of sexual dysfunction. In one embodiment, the (+) and (−) enantiomers of flosequinan were subjected to biochemical assays to determine their respective percent inhibition of PDE3. In one embodiment, it was shown that the (+) enantiomer of flosequinan has an eight-fold increase in PDE3 inhibition as compared to that of the (−) enantiomer of flosequinan at the same molar concentration. Moreover, in the same embodiment, the (+) enantiomer of flosequinan exhibited greater inhibition of PDE3 than a racemic mixture of flosequinan at the same molar concentration.

### DIAGNOSIS OF MALE ERECTILE DYSFUNCTION

Determination whether a human male is suffering from impotence that is substantially only neurogenic or psychogenic is readily made by a person skilled in the art using a number of readily available diagnostic procedures. Thus, a male suffering from impotence can first be given a physical examination with particular attention to possible penile and scrotal pathology, whereby any anatomical deficiency precluding an erection sufficient for vaginal penetration can be detected. In the absence of such an anatomical deficiency, the male can be subjected to tests, whereby penile venous leakage or severe or untreatable atherosclerosis can be detected.

Such tests include determination of the penobrachial blood pressure index (PBPI), doppler investigation of the penile arteries, and a papaverine test. The PBPI is the penile systolic blood pressure divided by the systolic blood pressure determined at one of the arms. These blood pressures can be determined by any number of standard techniques. Thus, the penile systolic blood pressure can be determined by i) placing an inflatable cuff around the base of the free part of the penis in the flaccid state which is capable of being used to apply variable pressure, readable from a gauge, to an object around which the cuff is placed, ii) localizing the penile arteries with a Doppler ultrasound probe (e.g., 8 MHz probe, such as the Mini Doplex D500 available from Huntleigh Technology, Luton, United Kingdom), and then iii) inflating and deflating the cuff and ascertaining the pressure at which the Doppler sound reappears.

The pressure at which the Doppler sound reappears is the penile systolic blood pressure. A male's penile blood pressure is regarded as normal if his PBPI is >0.80. With regard to Doppler investigation, each of the two penile cavernous arteries is investigated distal to the aforementioned cuff using the Doppler ultrasound problem. The function of each of the two arteries is assessed by Doppler ultrasound using an arbitrary scale of 0, 1, 2 or 3, where 0 means that the function is so deficient that the artery cannot be located and 3 means that the artery is well enough that maximal Doppler sound is observed.

In the papaverine test, a tourniquet is placed at the base of the free part of the penis and tightened and then, with the patient seated, 30 mg of papaverine in 1 ml of a physiologically acceptable fluid (e.g., physiological saline or phosphate-buffered saline) is injected into the penile cavernous body. In persons suspected of having impotence due to a suprasacral nerve lesion or a psychogenic dysfunction, only 15 mg of papaverine is administered, because of the high incidence of papaverine-induced priapism in such cases.

Five minutes after the injection, the tourniquet is removed and an ultrasound Doppler investigation of the penile cavernous arteries is carried out as described above. The function of the arteries is regarded as normal if both of them score a 3 on the arbitrary scale. After the Doppler investigation, penile vibration, at about a 4 Hz with an amplitude of about 1.2 mm (carried out with, e.g., a Vibrector, from Multicept, Gentofte, Denmark) is carried out for five to ten minutes and then erectile response is evaluated.

Erectile response is classified as full rigidity, if the angle between the penis and the legs in the standing position is >90°, and tumescence or no response if the angle is less than or equal to 45°. An impotent male, who does not have an anatomical deficiency that would preclude having an erection sufficient for vaginal penetration, who has a PBPI >0.80, who has scores of 2 or 3 in Doppler ultrasound investigations of both of the cavernous arteries of the penis, after papaverine injection as described above, and who has a fully rigid erection after papaverine injection and vibration as described above, is suffering from impotence that is “substantially only neurogenic or psychogenic” in origin.

It is possible that atherosclerosis or venous leakage contributes to such impotence, and atherosclerosis likely does contribute if the score is less than 3 in the Doppler investigation of one or both of the cavernous arteries after papaverine injection; but any venous leakage or atherosclerosis in such impotence is not untreatable and, consequently, is not a substantial factor in the impotence and such atherosclerosis, if any, is less than severe.

Impotence, which is a side-effect of drugs such as beta-blockers, is deemed to be neurogenic impotence in the present specification. Similarly, impotence which is a result of alcoholism or excessive consumption of alcohol, is deemed to be neurogenic or psychogenic impotence, for purposes of the present specification. Thus, a male who is diagnosed in accordance with the present specification as suffering from impotence that is “substantially only neurogenic or psychogenic” in origin is suffering from impotence that is substantially only neurogenic, psychogenic or neurogenic and psychogenic in origin, even though an underlying cause of the impotence has been identified as a side-effect of a drug, alcoholism or excessive consumption of alcohol.

Generally, a male with a PBPI less than about 0.60, with scores of 0 in Doppler investigations of both penile cavernous arteries (after papaverine injection as described above), and with a less than fully rigid erection after papaverine injection and vibration will have impotence caused by “untreatable” atherosclerosis. Methods are available to ascertain whether impotence is untreatable because of venous leakage.

One method of ascertaining whether untreatable venous leakage is a cause of impotence is by cavemosometry, optionally supplemented with cavernosography. (See, e.g., Delcour et al., Radiology 161: 799 (1986); Porst et al., J. Urol. 137: 1163 (1987); Lue et al., J. Urol. 37: 829 (1987)). Cavernosometry can be done using, both before and after intracavemosal injection of 60 mg of papaverine (in 1 ml of physiological saline), infusion of physiological saline through a 19-gauge needle into one corpus cavernosum with a 21-gauge needle inserted into the other corpus cavernosum for measurement of intracorporal pressure (which is recorded on a plotter).

The infusion rates needed to induce and maintain an erection are measured. If the infusion rate needed to maintain an erection is greater than 50 ml/min before administration of the papaverine and greater than 15 ml/min after administration of the papaverine, untreatable venous leakage is present. As long as an erection can be achieved at some flow rate less than about 100 ml/min before injection of the papaverine and less than about 50 ml/min after the injection of papaverine, it might be possible, using cavernosography, to locate the venous lesion associated with the leakage, and thereby confirm the diagnosis based on cavernosometry and provide information for possible surgical correction for the leakage. In the cavernosography, the penis is X-rayed, both before and after intracavernosal injection of 60 mg papaverine (in 1 ml of physiological saline), while infusing contrast medium into the corpus cavernosum (e.g., through a 19-gauge needle) at a flow rate that maintains an erection during the X-ray. Numerous contrast media suitable for the procedure are available in the art; these are typically aqueous solutions of iodinated compounds that provide between about 180 mg/ml and about 360 mg/ml of iodine. Examples are a solution of iohexol providing 240 mg/ml of iodine sold by Winthrop Pharmaceuticals, New York, N.Y., USA, and a solution of iopamidol providing 300 mg/ml iodine sold by Astra Meditec, Goteborg, Sweden. Typically 50-100 ml of the contrast medium will be employed for each x-ray (i.e., before and then after the injection of papaverine). In the cavernosometry and cavemosography, 30 mg papaverine (in 1 ml physiological saline) coupled with stimulation by vibration can be employed in place of 60 mg papaverine (in 1 ml physiological saline).

### DIAGNOSIS OF FEMALE ERECTILE DYSFUNCTION

Females have sexual dysfunction. Post-menopausal women often complain of discomfort with intercourse, dryness of the vagina and diminished vaginal arousal. Studies comparing sexual dysfunction in couples have revealed 40% of the men had erectile or ejaculatory dysfunction, whereas 63% of the women had arousal or orgasmic dysfunctions. Similar to male sexual dysfunction, the prevalence of female sexual dysfunction has been shown to increase with age and be associated with the presence of vascular risk factors and the development of the menopause.

The clitoris is the homologue of the penis. It is a cylindrical, erectile organ composed of the glans, corporal body and the crura. The corporal body is surrounded by a fibrous sheath, tunica albuginea, which encases cavernosal tissue consisting of sinusoids and surrounding smooth muscle. The clitoris responds to sexual excitement by tumescence and erection, although this does not occur with the degree of pressure elevation as found during penile erection. The characteristics of the clitoral blood flow, however, approximately parallel those of the male. See K. Park et al., “Vasculogenic female sexual dysfunction: The hemodynamic basis for vaginal engorgement insufficiency and clitoral erectile insufficiency,” Int. J. Impotence Res. 9:27 (1997).

Post-menopausal women and women with a history of vascular risk factors have been shown to have significantly more complaints of self-reported female vaginal and clitoral dysfunctions than pre-menopausal women or women without vascular risk factors. Such problems include, but are not limited to, atherosclerosis-induced vaginal engorgement insufficiency and clitoral erectile insufficiency syndromes.

Determination whether a human female is suffering from poor blood flow or supply is readily made by a person skilled in the art using a number of readily available diagnostic procedures. The human vagina receives arterial blood supply from the vaginal artery, the vaginal branch of the uterine artery, the internal pudendal artery, and the vaginal branches of the middle rectal artery. Blood flow in these areas can readily be assessed by a number of techniques. Arterial blood can be obtained and the blood levels of cholesterol and triglycerides can be analyzed as a first step. However, the preferred method is imaging.

While relatively non-invasive imaging is preferred, more invasive techniques can be used. For example, vaginal wall blood flow can be measured by laser Doppler flow probes placed into the vaginal muscularis layer within the spongy region of blood-filled spaces and vascular smooth muscle. Clitoral intracavernosal erectile tissue blood flow can be measured with a similar laser Doppler flow probe placed into the corporal bodies. The flow probes are connected to a laser Doppler flowmeter (Transonic Systems, Inc.) which is calibrated against an internal standard reading flow in units of ml/min/100 gm of tissue.

The laser Doppler probe uses the Doppler shift of a projected beam of laser light that registers on a photodetector. Static tissues will produce no Doppler shift in wavelength but moving red blood cells will produce a shift proportional to the red cell velocity.

### TREATMENT OF MALE AND FEMALE ERECTILE DYSFUNCTION

It is not intended that the present invention be limited by the particular nature of the therapeutic preparation. In one embodiment, the (+) enantiomer of flosequinan is provided together with physiologically tolerable liquid, gel or solid carriers, diluents, adjuvants and excipients. In addition, enantiomers of flosequinan may be used together with other chemotherapeutic agents. On the other hand, formulations may also contain such normally employed additives as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions typically contain 1%-95% of active ingredient, preferably 2%-70%.

The present invention is not limited by the method of introduction of the therapeutic compound to the body. Among other methods, the present invention contemplates administering cutaneously, orally, intracavemosally, transurethrally, by standard injection (e.g. intravenous or intramuscular), intranasally or through respiratory inhalation.

It is believed that oral administration of the (+) flosequinan enantiomer and pharmaceutical compositions comprising the (+) enantiomer of flosequinan is an effective mode of administration. Peak plasma concentrations of flosequinan (+) enantiomer are observed 1-2 hours following oral administration, while peak metabolite plasma levels are observed about seven hours following oral dosage. In one embodiment, the enantiomer of flosequinan is introduced into the male or female orally. It is believed that an oral dosage up to approximately 200 milligrams of a racemic mixture of flosequinan is an effective oral dosage. It is also believed that the oral administration of a purified of enantiomer of flosequinan is effective at even lower dosages (e.g. less than 200 mg). While the present invention is not limited to a specific dosage level, in one embodiment, the male or female is an adult human and the oral dosage of a purified enantiomer of flosequinan is in a single dose per day of up to approximately two hundred milligrams, more preferably, between approximately fifty to approximately seventy-five milligrams. In an even more preferred embodiment, a purified enantiomer of flosequinan is administered in a single oral dose per day of between approximately twenty and approximately fifty, and even more preferably, between approximately ten and approximately twenty milligrams per day.

The enantiomers of flosequinan are water soluble and are soluble in many organic solvents. Thus, while the present invention is not limited by the form of oral administration, aqueous and organic solutions of flosequinan enantiomer for oral administration are contemplated. Likewise, flosequinan enantiomers can be associated with a solid pharmaceutical carrier for solid oral administration (i.e., in pill form). One skilled in the art is able to readily prepare such solid formulations, and in one embodiment, the inactive ingredients include croscarmellose sodium, hydroxypropyl methylcellulose, lactose, magnesium stearate, methocel E5, microcrystalline cellulose, povidine, propylene glycol and titanium dioxide.

Flosequinan enantiomers and pharmaceutical compositions comprising flosequinan enantiomers may also be administered cutaneously in a carrier adapted for topical administration. Such carriers include creams, ointments, lotions, pastes, jellies, sprays, aerosols, bath oils, or other pharmaceutical carriers which accomplish direct contact between flosequinan and the pore of the skin. In general pharmaceutical preparations may comprise from about 0.001% to about 10%, and preferably from about 0.01 to 5% by w/w of the active compound (e.g., flosequinan) in a suitable carrier. In some cases it may be necessary to dissolve the flosequinan enantiomer or pharmaceutical compositions comprising a flosequinan enantiomer in an appropriate solvent such as ethanol or DMSO (dimethylsulfoxide), and the like, to facilitate incorporation into a pharmaceutical preparation. Likewise, the present invention can be incorporated in other products associated with sexual activity. For example, a coated, erection inducing condom as disclosed in U.S. Pat. No. 4,829,991, hereby incorporated by reference, can be utilized with flosequinan enantiomers or pharmaceutical compositions comprising flosequinan enantiomers.

While the present invention is not limited by a specific method of introducing flosequinan enantiomers, and pharmaceutical compositions thereof, injection of flosequinan enantiomers, or a pharmaceutical composition thereof can be carried out by any conventional injection means (e.g., employing an hypodermic syringe and needle or a similar device such as the NovolinPen. sold by Squibb-Novo, Inc., Princeton, N.J., USA). This injection may be by subject injecting himself or by another person (such as a partner during sexual relations or a physician prior to sexual relations) injecting the male whose erection is to be induced. In one embodiment, flosequinan enantiomers are introduced intracavemously as described in U.S. Pat. No. 5,447,912 to Gerstenberg et al., hereby incorporated by reference.

Flosequinan enantiomers and pharmaceutical compositions comprising flosequinan enantiomers can be introduced intracavemosally in a physiologically acceptable composition. Such compositions are aqueous solutions that are physiologically acceptable for administration by intracavernosal injection into the penis. The physiologically acceptable carrier is selected such that it is not painful or irritating upon intracavernosal injection. The physiologically acceptable compositions will preferably be sterile at the time of administration by intracavernosal injection.

Among the physiologically acceptable compositions for use in the methods is physiological saline or phosphate buffered saline, in which flosequinan enantiomers or a pharmaceutical composition comprising enantiomers of flosequinan, is dissolved or suspended, such that the resulting composition is suitable for intracavernosal injection. Such a physiologically acceptable composition can also include a non-irritant preservative, such as, e.g., benzalkonium chloride at 0.05% (w/v) to 0./2% (w/v). As the skilled artisan will understand, there are numerous non-toxic salts of VIP, PHM and α-adrenergic blockers that can be employed in a physiologically acceptable composition for use in the methods herein, including, among others, the chloride, bromide, acetate, sulfate, and mesylate salts.

In carrying out the methods, it is preferred that, for a period of time between about 1 minute and about 15 minutes (preferably about 5 minutes-10 minutes), the penis is constricted near the base thereof and between the base and the point at which the injection into a corpus cavernosum occurs, in order to limit loss of injected fluid from the corpus cavemosum before the ingredients in the fluid, that are active in inducing erection, have been able to have erection-inducing effects. The constriction can be effected by any means known in the art, such as with a tourniquet, cuff, rubber band or the like, or even manually, in order to slow the release of the injected fluid and the pharmacologically active substance(s) therein into the general circulation.

Likewise, the present invention is not limited by a particular method for introducing flosequinan enantiomers or a pharmaceutical composition comprising enantiomers of flosequinan transurethrally. In one embodiment, the (+) flosequinan enantiomer, or a pharmaceutical composition thereof, is introduced to the urethra in a carrier as described for cutaneous administration. Devices and methods for transurethral introduction of pharmaceutical compositions is described in U.S. Pat. No. 5,474,535 to Place et al.; Voss, U.S. Pat. No. 4,801,587 and Kock, EPA 0357581, all hereby incorporated by reference.

Additional methods of the transurethral introduction of flosequinan enantiomers, or pharmaceutical compositions thereof, include the use of medicated catheters, such as those used to prevent or treat localized infections and irritation of the urethra and bladder (See U.S. Pat. No. 4,640,912, hereby incorporated by reference). Alternatively, transurethral administration of pharmaceutical compositions is presented in U.S. Pat. Nos. 4,478,822, 4,610,868, 4,640,912 and 4,746,508, all hereby incorporated by reference, and medicated urethral suppositories, inserts or plugs, typically containing anti-infective agents or spermicide are disclosed in U.S. Pat. Nos. 1,897,423, 2,584,166, 2,696,209 and 3,373,746, all incorporated by reference.

While the present invention is not limited to the method of injecting enantiomers of flosequinan, or pharmaceutical compositions thereof, in the preferred embodiment, flosequinan or a pharmaceutical composition comprising flosequinan is injected with a standard syringe. One skilled in the art would be capable of injecting flosequinan or a pharmaceutical composition comprising flosequinan with a carrier as described for intracavernosal injection.

Flosequinan enantiomers and pharmaceutical compositions comprising flosequinan enantiomers may also be administered intranasally. Formulations suitable for intranasal administration include ointments, creams, lotions, pastes, gels, sprays, aerosols, oils and other pharmaceutical carriers which accomplish direct contact between flosequinan or a pharmaceutical composition comprising flosequinan and the nasal cavity. Examples of pharmaceutical compositions administered intranasally are described in U.S. Pat. Nos. 5,393,773 and 5,554,639 to Craig et al.; and 5,801,161 to Merkus, all hereby incorporated by reference.

Flosequinan enantiomers and pharmaceutical compositions comprising flosequinan enantiomers may also be administered through respiratory inhalation. Formulations suitable for respiratory inhalation include ointments, creams, lotions, pastes, gels, sprays, aerosols, oils and other pharmaceutical carriers which accomplish direct contact between flosequinan enantiomers or a pharmaceutical composition comprising flosequinan enantiomers and the respiratory tract. Examples of pharmaceutical compositions administered through respiratory inhalation are described in U.S. Pat. Nos. 4,552,891 to Hu et al.; 5,869,479 to Kreutner et al., and 5,864,037 to Chasis et al.

In some embodiments, intranasal administration and respiratory inhalation are the preferred modes of administration due to the ease of administration and faster onset of therapeutic activity. It is contemplated that intranasal administration and respiratory inhalation are advantageous as they may allow a smaller effective dosage to be administered than would be possible with the oral route of administration. A preferred mode of administration comprises administration to the lung. Intrapulmonary delivery of pharmacologic agents to patients can be accomplished via aerosolization. Alternatively, the agent may be administered to the lung through a bronchoscope. Of course, the therapeutic agents may be investigated for their efficacy via other routes of administration, including parenteral administration.

In one embodiment, the administration of the compositions of the present invention is accompanied by sexual stimulation to induce an erection. The sexual stimulation can begin before or after the introduction of flosequinan or a pharmaceutical composition comprising flosequinan. If the stimulation begins after the injection, it is preferably begun within 5 to 10 minutes to insure that there is significant overlap of the pharmacological effects of the pharmaceutical composition administered and the stimulative effects of the sexual stimulation. Whether the stimulation begins before or after the injection, it will continue preferably at least until an erection sufficient for vaginal penetration is achieved.

Sexual stimulation as prescribed by these methods, includes any form of sexual stimulation that would induce an erection in a normal male who is not suffering from erectile insufficiency. The sexual stimulation can be that which occurs in the course of sexual relations between the subject and another person or can be outside sexual relations with another person. Examples of methods of sexual stimulation include, alone or in combination, touching or erotically manipulating erogenous areas of the genital organs or other erogenous parts of the body; providing visual stimulation, as with a sexually explicit media (e.g., pornographic film) or other form of sexually stimulative show or display. Additionally, providing vibratory stimulation to the penis, at between about 30 Hz and about 100 Hz with an amplitude of about 1 mm to about 5 ram, as can be provided, for example, by resting the penis on the table of a vibrating apparatus such as that of a Vibrector system (Multicept, Genofte, Denmark).

In inducing an erection in an impotent male outside of sexual relations, as, for example, when a physician induces an erection in a patient suffering from psychogenic impotence, a preferred method of sexual stimulation includes providing visual stimulation, as with a pornographic film, simultaneously with vibratory stimulation of the penis, as with a Vibrector system set to between about 30 Hz and about 60 Hz (usually about 50 Hz)in frequency and between about 1 mm and about 2.5 mm (usually about 2.2 mm) in amplitude.

From the above, it should be clear that the present invention provides methods of treatment of sexual dysfunction (e.g. erectile dysfunction) with pharmaceutical agents. In particular, flosequinan enantiomers, or pharmaceutical compositions comprising flosequinan enantiomers, are administered therapeutically to patients having such dysfunction.

### EXPERIMENTAL

The following examples serve to illustrate certain preferred embodiments and aspects of the present invention and are not to be construed as limiting the scope thereof.

In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); μg (micrograms); L (liters); ml (milliliters); μl (microliters); cm (centimeters); mm (millimeters); μm (micrometers); nm (nanometers); ° C. (degrees Centigrade).

### EXAMPLE 1

In this example, a biochemical assay was performed to test the percentage of phosphodiesterase 6 (PDE6) inhibition of various molar concentrations of sildenafil citrate (Viagra) as compared to that of a 100 μM concentration of a racemic mixture of flosequinan as follows.

PDE6 partially purified from bovine retinal rod and activated by trypsin was used. In four separate reactions, Viagra at molar concentrations of 0.3 μM, 1.0 μM, and 3.0 μM, and a 100 μM racemic mixture of flosequinan were incubated with 0.2 μg/ml active PDE6 and 100 μM cGMP containing 0.1 μM [³H]cGMP in Tris buffer pH 7.5 for 20 minutes at 30° C. Each reaction was terminated by raising the temperature to 100° C. for 2 minutes. The resulting GMP was converted to guanosine by addition of 10 mg/ml snake venom nucleotidase and further incubated at 30° C. for 10 minutes. Unhydrolyzed cGMP was bound to AGI-X2 resin, and [³H]guanosine remaining in the aqueous phase was quantitated by scintillation counting.

The results of the assays, as noted in the table below, indicate that although Viagra inhibits PDE6 around 50% at concentrations as low as 0.3 μM, such levels of inhibition of PDE6 require greater than 100 μM amounts of flosequinan (i.e. more than 300 times more compound on a molar basis). These empirical results could not be predicted.

| Compound | Concentration | % Inhibition of PDE6 |
|---|---|---|
| Viagra | 3.0 μM | 87 |
| Viagra | 1.0 μM | 62 |
| Viagra | 0.3 μM | 57 |
| Flosequinan | 100 μM | 36 |

### EXAMPLE 2

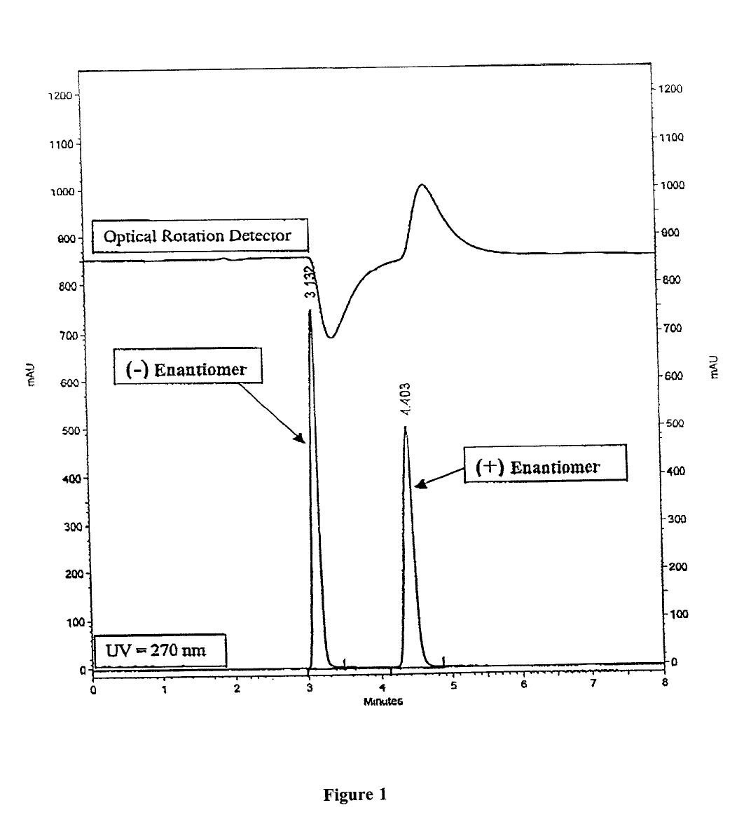

In this example, the enantiomers of flosequinan were resolved by high-performance liquid chromatography (HPLC) as follows. A 5.0 g sample of a racemic mixture of flosequinan was resolved over a 10 cm ID×50 cm L CHIRALCEL OD HPLC column (Chiral Technologies, Exton, Pa.) at 25° C. and with a flow rate of 1.0 ml/minute such that the column pressure was 37 bar. The mobile phase employed was 100% methanol and the detection of the mixture was performed at 270 nm. The (−) enantiomer had a retention time of 3.13 minutes, while the (+) enantiomer had a retention time of 4.40 minutes. A total of 2.1 g of the (−) enantiomer having an optical purity greater than 99% was produced. A total of 2.3 g of the (+) enantiomer having an optical purity greater than 99% was produced. (See FIG. 1).

### EXAMPLE 3

In this example, a racemic mixture of flosequinan and the (+) and (−) enantiomers of flosequinan were subjected to biochemical enzyme assays to determine their respective percent inhibition of a variety of phosphodiesterases (PDE1-PDE6). The reaction conditions for each PDE assay were as follows.

PDE1: PDE1 partially purified from bovine heart was used. A racemic mixture of flosequinan, and each enantiomer of flosequinan, all at a molar concentration of 100 μM, were independently incubated with 13 μg PDE1 enzyme, 1.0 μM [³H]cAMP and CaCl₂/calmodulin in Tris buffer pH 7.5 for 20 minutes at 30° C. The reaction was terminated by boiling for 2 minutes, and the resulting AMP was converted to adenosine by addition of 10 mg/ml snake venom nucleotidase and further incubation at 30° C. for 10 minutes. Unhydrolyzed cAMP was bound to AGI-X2 resin, and the remaining [³H]adenosine in the aqueous phase was quantitated by scintillation counting.

PDE2: PDE2 partially purified from human platelets was used. A racemic mixture of flosequinan, and each enantiomer of flosequinan, all at a molar concentration of 100 μM, were independently incubated with 23 μg PDE2 enzyme, 25 μM cAMP containing 0.05 μM [³H]cAMP in Tris buffer pH 7.5 for 20 minutes at 30° C. The reaction was terminated by boiling for 2 minutes, and the resulting AMP was converted to adenosine by addition of 10 mg/ml snake venom nucleotidase and further incubation at 30° C. for 10 minutes. Unhydrolyzed cAMP was bound to AGI-X2 resin, and the remaining [³H]adenosine in the aqueous phase was quantitated by scintillation counting.

PDE3: PDE3 partially purified from human platelets was used. A racemic mixture of flosequinan, and each enantiomer of flosequinan, all at a molar concentration of 100 μM, were independently incubated with 13 μg PDE3 enzyme and 1 μM cAMP containing 0.01 μM [³H]cAMP in Tris buffer pH 7.5 for 20 minutes at 30° C. The reaction was terminated by boiling for 2 minutes, and the resulting AMP was converted to adenosine by addition of 10 mg/ml snake venom nucleotidase and further incubation at 30° C. for 10 minutes. Unhydrolyzed cAMP was bound to AGI-X2 resin, and the remaining [³H]adenosine in the aqueous phase was quantitated by scintillation counting.

PDE4: PDE4 partially purified from human U-937 pronocytic cells was used. A racemic mixture of flosequinan, and each enantiomer of flosequinan, all at a molar concentration of 100 μM, were independently incubated with 20 μg PDE4 enzyme and 1 μM cAMP containing 0.01 μM [³H]cAMP in Tris buffer pH 7.5 for 20 minutes at 30° C. The reaction was terminated by boiling for 2 minutes, and the resulting AMP was converted to adenosine by addition of 10 mg/ml snake venom nucleotidase and further incubation at 30° C. for 10 minutes. Unhydrolyzed cAMP was bound to AGI-X2 resin, and the remaining [³H]adenosine in the aqueous phase was quantitated by scintillation counting.

PDE5: PDE5 partially purified from human platelets was used. A racemic mixture of flosequinan, and each enantiomer of flosequinan, all at a molar concentration of 100 μM, were independently incubated with 120 μg PDE5 enzyme and 1 μM cGMP containing 0.01 μM [³H]cGMP in Tris buffer pH 7.5 for 20 minutes at 30° C. The reaction was terminated by boiling for 2 minutes, and the resulting GMP was converted to guanosine by addition of 10 mg/ml snake venom nucleotidase and further incubation at 30° C. for 10 minutes. Unhydrolyzed cGMP was bound to AGI-X2 resin, and the remaining [³H]guanosine in the aqueous phase was quantitated by scintillation counting.

PDE6: PDE6 partially purified from bovine retinal rod and activated by trypsin was used. A racemic mixture of flosequinan, and each enantiomer of flosequinan, all at a molar concentration of 100 μM, were independently incubated with 0.2 μg/ml active PDE6 and 100 μM cGMP containing 0.1 μM [³H]cGMP in Tris buffer pH 7.5 for 20 minutes at 30° C. Each reaction was terminated by boiling for 2 minutes. The resulting GMP was converted to guanosine by addition of 10 mg/ml snake venom nucleotidase, and further incubated at 30° C. for 10 minutes. Unhydrolyzed cGMP was bound to AGI-X2 resin, and [³H]guanosine remaining in the aqueous phase was quantitated by scintillation counting.

The results of the above PDE assays are presented in the following table. The results indicate that the (+) enantiomer of flosequinan demonstrated more PDE1 and PDE3 inhibitory activity when compared with the (−) enantiomer of flosequinan. These empirical results could not be predicted.

| | % Inhibition w/ | | |
|---|---|---|---|
| | 100 μM racemic | % Inhibition w/ | % Inhibition w/ |
| Target | mixture of | 100 μM (+)- | 100 μM (−)- |
| Phosphodiesterase | flosequinan | flosequinan | flosequinan |
| PDE1 | 31 | 28 | 11 |
| PDE2 | 18 | 18 | 13 |
| PDE3 | 26 | 32 | 5 |
| PDE4 | 24 | 6 | 1 |
| PDE5 | 11 | 17 | 10 |
| PDE6 | 21 | 22 | 21 |

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

We claim:

1. A method, comprising:
a) providing:
i) a patient suffering from symptoms of sexual dysfunction, and
ii) a purified enantiomer preparation of flosequinan, or a pharmaceutically acceptable salt thereof; and
b) administering said preparation to said patient such that such symptoms are reduced.

2. The method of claim 1, wherein said purified enantiomer preparation of flosequinan is a (+) enantiomer.

3. The method of claim 1, wherein said preparation is substantially free of the (−) enantiomer of flosequinan.

4. The method of claim 1, wherein said patient is a male.

5. The method of claim 1, wherein said patient is a female.

6. The method of claim 1, wherein said patient is free from cardiac disease.

7. The method of claim 1, wherein said administering step is selected from the group consisting of intranasal administration and respiratory inhalation.

8. A method, comprising:
a) providing:
i) a patient suffering from symptoms of sexual dysfunction who is not being treated with a drug that causes hypotensive effects, and
ii) a purified enantiomer preparation of flosequinan, or a pharmaceutically acceptable salt thereof; and
b) administering said preparation to said patient such that such symptoms are reduced.

9. The method of claim 8, wherein said administering step is selected from the group consisting of intranasal administration and respiratory inhalation.

10. The method of claim 8 wherein, said patient has not been treated in the past with a drug that causes hypotensive effects.

11. The method of claim 8, wherein said purified enantiomer preparation of flosequinan is a (+) enantiomer.

12. The method of claim 8, wherein said preparation is substantially free of the (−) enantiomer of flosequinan.

13. The method of claim 8, wherein said patient is a male.

14. The method of claim 8, wherein said patient is a female.

15. A method, comprising:
a) providing:
i) a patient suffering from symptoms of sexual dysfunction who is not being treated with a nitrite or nitrate, and
ii) a purified enantiomer preparation of flosequinan, or a pharmaceutically acceptable salt thereof; and
b) administering said preparation to said patient such that such symptoms are reduced.

16. The method of claim 15, wherein said administering step is selected from the group consisting of intranasal administration and respiratory inhalation.

17. The method of claim 15, wherein said nitrate is selected from the group consisting of glyceryl trinitrate, isosorbide dinitrate, isosorbide-5′-mononitrate and erythrityl tetranitrate.

18. The method of claim 15, wherein said purified enantiomer preparation of flosequinan is a (+) enantiomer.

19. The method of claim 15, wherein said preparation is substantially free of the (−) enantiomer of flosequinan.

20. The method of claim 15, wherein said patient is a male.

21. The method of claim 15, wherein said patient is a female.
